(19) United States
(12) Patent Application Publication
(10) Pub. No.: US 2025/0246290 A1
(43) Pub. Date: Jul. 31, 2025

(54) NETWORKED RESPONSIVE PERSONAL GUIDANCE SYSTEM FOR KNOWN CONDITIONS

(71) Applicant: David Bianchi, Wimberley, TX (US)

(72) Inventor: David Bianchi, Wimberley, TX (US)

(21) Appl. No.: 19/038,196

(22) Filed: Jan. 27, 2025

Related U.S. Application Data
(60) Provisional application No. 63/625,147, filed on Jan. 25, 2024.

(51) Int. Cl. G16H 20/70; G06F 40/30; G16H 50/30; G16H 80/00

(52) U.S. Cl. CPC G16H 20/70; G06F 40/30; G16H 50/30; G16H 80/00

(57) ABSTRACT

An indication of a known condition of a user is received in a computerized system. The system is operable to monitor the user for one or more physical indications related to the known condition, and provide counseling for the known condition to the user responsive to observation of the one or more monitored physical indications via a networked responsive personal guidance system trained on the condition. The networked responsive personal guidance system may be adapted based on the received input from the user, such as to remember a state of prior interactions with the user to provide consistent conversational guidance across multiple login sessions for the user.

### CLAIM OF PRIORITY

This application claims the benefit of priority to U.S. Provisional Application Ser. No. 63/625,147, titled “NETWORKED PERSONAL GUIDANCE SYSTEM USING ARTIFICIAL INTELLIGENCE,” filed on Jan. 25, 2024, and incorporated herein by reference in its entirety.

### FIELD

The field relates generally to personal guidance or counseling, and more specifically to a networked responsive personal guidance system.

### BACKGROUND

Computers are valuable tools in large part for their ability to communicate with other computer systems and retrieve information over computer networks. Networks typically comprise an interconnected group of computers, linked by wire, fiber optic, radio, or other data transmission means, to provide the computers with the ability to transfer information from computer to computer. The Internet is perhaps the best-known computer network, and enables millions of people to access millions of other computers such as by viewing web pages, sending e-mail, or by performing other computer-to-computer communication.

Computer devices interconnected via networks have evolved over time form simple standalone computers such as the traditional personal computer to smart set-top boxes, computerized smart home controllers, and smart phones having capabilities that rival the most complex computers of a generation ago. Modern smart phones often include large display screens, cameras, microphones and speakers, and a variety of other components that enable them to perform a wide variety of functions, such as web browsing, voice or video communication, Global Positioning System (GPS) navigation, and the like. Some applications or apps, such as exercise trackers, weight loss trackers, and the like may use user inputs to help track health, exercise, or similar functions, and to track progress toward a personal goal.

Smart phone apps are also often used to interface with medical providers where actual medical advice is needed (often called telemedicine), such as to provide a medical diagnosis, a recommendation for treatment, or a prescription for medicine. Telemedicine may eliminate the need to schedule an appointment with a medical provider in advance, and can provide treatment with a medical provider matched to the user's medical condition that is much more cost-effective than an in-person appointment at a clinic.

But, telemedicine providers are typically not engaged with the same client long-term and do not know the client well, and often do not have access to a person's full medical history. Telemedicine also often still involves the expense of paying a medical provider to provide medical diagnosis, treatment, or prescriptions, and may be fairly expensive and time-consuming for complex conditions such as mental health counseling. A need therefore exists for improved automated guidance or counseling.

### BRIEF DESCRIPTION OF THE DRAWINGS

The claims provided in this application are not limited by the examples provided in the specification or drawings, but their organization and/or method of operation, together with features, and/or advantages may be best understood by reference to the examples provided in the following detailed description and in the drawings, in which:

FIG. 1 is a block diagram of a networked system of computerized devices, as may be used to practice some embodiments.

FIG. 2 is a flow diagram showing user interaction with a persona guidance system, consistent with an example embodiment.

FIG. 3 is a flow diagram of a method of providing guidance to a user, consistent with an example embodiment.

FIG. 4 shows a block diagram of a generative pretrained transformer, as may be used to practice some embodiments.

FIG. 5 shows a block diagram of a general-purpose computerized system, consistent with an example embodiment.

Reference is made in the following detailed description to accompanying drawings, which form a part hereof, wherein like numerals may designate like parts throughout that are corresponding and/or analogous. The figures have not necessarily been drawn to scale, such as for simplicity and/or clarity of illustration. For example, dimensions of some aspects may be exaggerated relative to others. Other embodiments may be utilized, and structural and/or other changes may be made without departing from what is claimed. Directions and/or references, for example, such as up, down, top, bottom, and so on, may be used to facilitate discussion of drawings and are not intended to restrict application of claimed subject matter. The following detailed description therefore does not limit the claimed subject matter and/or equivalents.

### DETAILED DESCRIPTION

In the following detailed description of example embodiments, reference is made to specific example embodiments by way of drawings and illustrations. These examples are described in sufficient detail to enable those skilled in the art to practice what is described, and serve to illustrate how elements of these examples may be applied to various purposes or embodiments. Other embodiments exist, and logical, mechanical, electrical, and other changes may be made.

Features or limitations of various embodiments described herein, however important to the example embodiments in which they are incorporated, do not limit other embodiments, and any reference to the elements, operation, and application of the examples serve only to aid in understanding these example embodiments. Features or elements shown in various examples described herein can be combined in ways other than shown in the examples, and any such combinations is explicitly contemplated to be within the scope of the examples presented here. The following detailed description does not, therefore, limit the scope of what is claimed.

Computing devices such as smart phones have evolved to include cameras, large display screens, video capability, and other such hardware features that facilitate use for applications such as teleconferencing and augmented reality in addition to traditional uses such as checking email, browsing the Internet, and playing games. These capabilities have contributed to a rapid growth in applications or apps that leverage such hardware, such as telehealth apps in which a user can request a consultation with a healthcare provider via a teleconference rather than schedule and wait for a traditional appointment at a clinic or hospital.

Telehealth app-based visits may be limited to visits for conditions that can be treated over the phone, such as colds, sore throats, skin conditions, and the like, and may generally not be well-suited to applications where more complex diagnosis methods need to be employed such as examining an ear with an otoscope for an ear infection. The time it takes to conduct a telehealth visit may be brief relative to a clinic visit, resulting in a significant cost savings and convenience for both the user and the health care provider.

Some telehealth providers may provide counseling or mental health services via telehealth, but such visits may be longer in length due to the nature of care being provided and so any cost savings may be significantly reduced. Telehealth providers may also not have a complete medical history of a patient and may not know the patient in advance, but may be assigned to a user based on the user's place in a queue and on limited additional factors such as the type of care being sought. Telehealth apps may therefore provide limited benefits or cost savings over in-person counseling or guidance for issues such as mental health, and may provide guidance that does not adequately take into account a person's relatively complex mental health history, personal relationships and background affecting their condition, and other such factors.

Some examples presented herein therefore provide for improved mental health, counseling, and well-being for a user, using methods such as artificial intelligence to learn about a user's condition and provide appropriate guidance and support to a user. In a more detailed example, a large language model such as Grok, ChatGPT, or the like may be trained to recognize a variety of psychological conditions, mental health issues, and the like, and to provide an appropriate response when presented with a question related to such issues. A user's personal history, relationships, medical history, and the like may be further learned as part of interacting with the large language model, such as by training the large language model with such information or including such information in a prompt, that the large language model's responses are tailored to a particular user and to their unique circumstances or history. In some examples, the factual basis for the advice or information provided may further be provided, such as a reference or resource upon which the advice is derived.

The guidance provided via the large language model may be structured to be neutral to a person's background or beliefs, such as religion, politics, sexual orientation, or the like, and in other examples may be derived with sensitivity to the user's beliefs taken into account. A user's age and maturity level may similarly be taken into account in providing a response, as may a user's general intelligence or ability to quickly grasp and apply guidance to their own personal conditions.

In one embodiment referred to as “Jim Mini,” a large language model artificial intelligence may present itself as a guidance companion, such as in the following example:
Your Source for Life, Mental Health, and Well-Being Advice-Simply ask JIM-MINI Your personal AI Guidance companion.
I exist, and I'm ready for trial online exclusively for registered donors! Help me to find my way Into the hearts and home of every individual who truly need me. I am looking for funding so I can eliminate our battles with mental health and achieve overall well-being with the press of a button. Achieving this goal, we can then start funding to move the project to become available for all mobile devices not just iOS but android as well. Donors will have access to an Exclusive Beta test run.
I would like to welcome you to our community, your trusted resource for valuable advice on life, mental health, and overall well-being. We are committed to providing you with factual and unbiased information, regardless of your background, beliefs, or identity. Tailored to your age and maturity.

Our Mission:
To empower individuals with knowledge that can improve their lives.
To offer evidence-based guidance on mental health and well-being.
To support your journey towards a healthier and happier life.

What We Offer:
1. Life Advice: Explore practical tips and insights to enhance various aspects of your life, from personal development to relationships and career success.
2. Mental Health Insights: Access information on mental health conditions, coping strategies, and resources to help you or your loved ones navigate mental health challenges.
3. Well-Being Guidance: Discover ways to prioritize your well-being, including nutrition, fitness, stress management, and self-care practices.

Our Commitment:
We are dedicated to providing accurate and up-to-date information.
We respect your individuality and offer content that is inclusive and unbiased.
We do not discriminate based on religion, sex, or gender, and we welcome everyone seeking knowledge and support.
We will be offering our chat engines, your local events, and forums in all languages. Ensuring you get the help that you need with Ease.
Join us on your journey to a healthier, happier, and more fulfilling life,

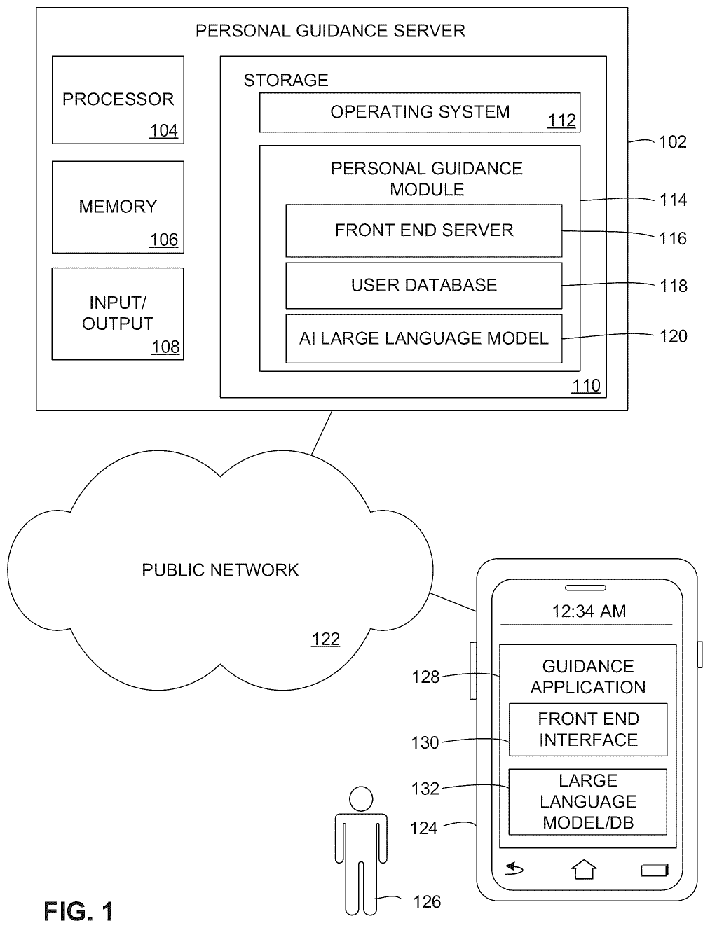

FIG. 1 is a block diagram of a networked system of computerized devices, as may be used to practice some embodiments. Here, a personal guidance server 102 comprises a processor 104, memory 106, input/output elements 108, and storage 110. Storage 110 includes an operating system 112 and personal guidance module 114 that is operable to provide personal guidance to one or more users via large language model artificial intelligence. The personal guidance module 114 in this example further comprises a front end server 116 operable to receive and service requests from client devices for guidance, social interaction, user registration or login, and the like. User database 118 comprises user information gathered from user registration, created and/or shared user content, and user information imported from other sources such as social media sites. AI large language model 120 is trained to recognize and respond to a variety of psychological, mental well-being, and other personal wellness topics such as physical health and self-regulation, and may generate a user-readable conversational response in response to a user prompt.

The personal guidance server 102 is coupled to a public network 122, such as the Internet, facilitating communication with one or more user devices such as smart phone 124 that user 126 employs to interact with the personal guidance server. Smart phone 124 in this example comprises a guidance application 128 such as may be installed from an app store or via other means, including a front end interface 130 and optionally a large language model and/or a user database 132 that may perform some or all of the functions of user database 118 and AI large language model 120 such as when the personal guidance server is unavailable.

In operation, a user initiates contact with the personal guidance server such as through installing an app, visiting a URL directing the user to a web page served at least in part by front end server 116, or through other suitable means. A new user is prompted to build a user profile that may be retained in user database 118, and may in a further example be used to train AI large language model 120, to train a user-specific version of the large language model, or to remember the state of prior user interactions with the AI large language model such that the large language model may provide consistent conversational guidance to the user across multiple login sessions. The user may in further examples enable real-time monitoring of the user's condition via the app, such as by using a smart watch or similar device operable to perform one or more biometric measurements of the user's physical state. Registration in some examples will allow or require entering emergency contact information, and/or consenting to being contacted by a live person should the guidance application 128 and/or the personal guidance server 102 determine that the user is at risk.

The user may in some embodiments choose to make one or more profiles, choose to make the profile(s) anonymous or identifiable, and may elect to leave out certain elements of their personal history. Demographic questions such as language, country, state, city, culture, race, ethnic background, beliefs, identity, age, and maturity level may be gathered to create a profile of the user, which in some further examples may be used to tailor artificial intelligence-based interaction with the user. Such parameters may be changed, such as where a user grows older and more mature, moves geographically, or the like. In some examples, parental consent for users who are underage may be obtained, and/or verification of parameters such as age may be performed.

Once a user profile is created, the user may log in and interact with the personal guidance server 102, such as by engaging in social interaction with other members that may have similar interests or be facing similar challenges, by interacting with the AI large language module to receive personal guidance, or by creating, sharing, or uploading content such as relevant media. A user interface may provide for interaction with the large language model 120 (or 132) in a conversational manner, such that a user may explain their struggles with mental health or other aspects of well-being, and discuss practical guidance for improving their life such as personal development, relationship guidance, and career success. In a further example, information provided to a user may be filtered or processed to remove bias and improve inclusivity. Further areas of personal guidance may include nutrition, fitness, stress management, self-care practices such as meditation, and the like. In a further example, the user and/or the personal guidance system may log the user's condition, progress, interactions, or the like, such as by using database 118 (or 132).

The user in some examples may create content, such as for social interaction with other users, to document their struggles, their coping mechanisms, or for other reasons. The content in various examples may comprise photos, videos, comments, groups, events, posts, and the like, and in a further example may comprise a marketplace-type posting offering goods or services. The content may be posted publicly or retained privately, and may be included in a news feed of other user such as friends, members of the same group, members sharing an interest or problem with the creating user, etc., Created content may also be sent via chat, mail, personal message, group message, and the like to one or more other users or groups.

The personal guidance module 114 in a further example comprises data processing functionality operable to apply algorithms for content distribution, moderation, personalization, and the like, and may be flagged for further review for compliance with standards of participation and/or sharing with others. The processed content may be stored in user database 118, along with user profiles, posts, media, social graph data, third-party advertisements, and other such data. Processed content may be retrieved in operation by front end server 116 or other data processing elements to present content to each user based on their preferences, settings, applicable algorithms, and the like. Users may then interact with content, engage with other users, and record likes, shares, and messages or responses to the content.

Personal guidance server 102 in further examples comprises other functions such as backend services to handle friend requests, messaging, content delivery, and the like, and may include various security or privacy measures such as encryption, access control, and monitoring to provide a degree of user privacy and security. The personal guidance module may comprise various additional functions or algorithms in various embodiments, such as algorithms for content recommendation, sentiment analysis, user behavior prediction, real-time user monitoring, and the like, such as to improve the personal guidance server's interaction with individual users.

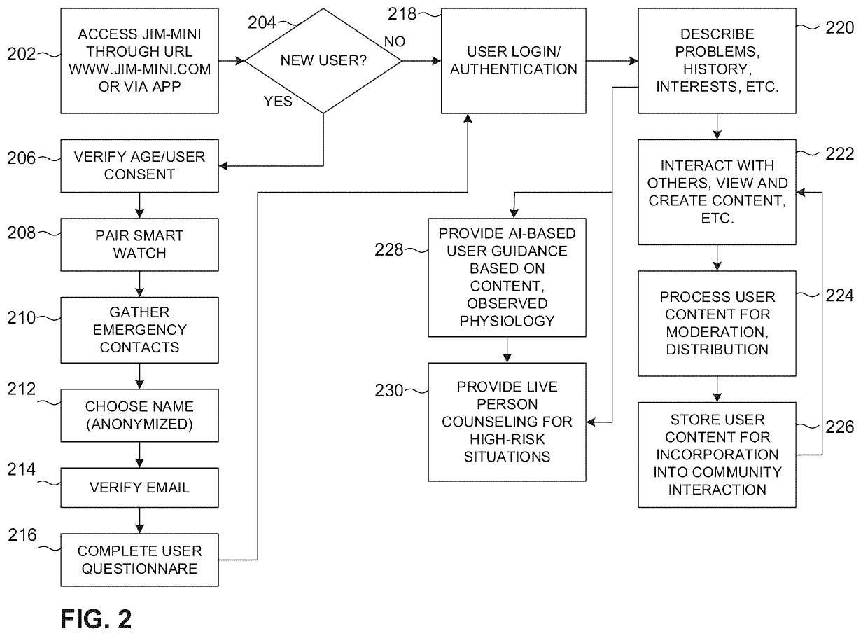

FIG. 2 is a flow diagram showing user interaction with a personal guidance system, consistent with an example embodiment. At 202, a user initiates a session with the personal guidance system, which in this example is named Jim-Mini. The user may initiate the session by visiting a website such as by entering the URL www.jim-mini.com in a web browser, or by downloading and executing an app such as on a smart phone or tablet.

The process determines whether the user is a new user at 204, and if the user is new, proceeds with a registration process shown at 206-216. The registration process starts at 206 with verification of the user's age and consent to the terms of use of the personal guidance system, and if the user is too young to consent to the terms, allows a parent or guardian to consent on the minor's behalf. The user is presented the option to pair a smart watch or other device having biometric monitoring functionality operable to monitor one or more biometrics at 208, such as a heart rate monitor, motion monitor, temperature monitor, blood oxygen monitor, stress monitor, or similar biometric monitor. The user may also be prompted to enter one or more emergency contacts at 210, such as a phone number and identity of a trusted friend or relative, and in a further example may be asked to consent to contacting the emergency contact and disclosing certain information such as location, condition, and the like should an emergency arise.

The user may be asked to choose a user name at 212, which in various examples may be an anonymous user name, the user's real name, or left to the user to decide. The user's email address may be verified at 214, such as by asking the user to enter their email address and sending a verification email to the user's provided address. The email contains a link that the user can click to verify that they are the owners of the email address, such as by reporting back to personal guidance system 102 that the user has clicked the unique link provided to them and associated with verifying the email address associated with their profile. The user may be asked to complete a user questionnaire at 216, entering information such as their mental health history, physical condition, goals, and the like, as well as demographic questions such as language, country, state, city, culture, race, ethnic background, beliefs, identity, age, and maturity level. In some examples, one or more of the questions may be optional, enabling the user to choose not to share select information they are not comfortable sharing in creating their profile. Once the user profile is completed, the user may be prompted to log in at 218 as though they are a returning user, or optionally may be automatically logged in and proceed to a later step such as is shown at 220.

If the user is a returning user who has already registered, the user may be prompted to log in at 218, such as by entering a username or email address along with a password or other verification such as a face ID, fingerprint, or the like. Once the user is logged in, they may have an opportunity to perform any of a variety of selected actions, including those shown at 220-230 and/or other such functions as they choose. In the example of FIG. 2, the user is prompted to enter information regarding their problems such as mental or physical problems, history such as relationship, work, or other mental health history relevant to their problems or goals, and interests or goals such as improving stress management, having more successful relationships, or losing weight.

The user is presented the opportunity to interact with other users at 222, such as by making posts to a news feed, chatting in a chat room, sending messages or email, creating content, and the like. The user may similarly view these interactions originated by other users, such as by checking messages, browsing a news feed, or responding to others in a chat room. At 224, certain user-created content may be processed for moderation prior to distribution such as inclusion in others' news feeds or inclusion in instant messages or chat room chats, such as to ensure that the content is appropriate for the given environment and does not violate terms of use or other rules. The processed user content may be stored at 226, such as in user database 118 of FIG. 1, for incorporation into such community interactions.

The user may also choose to interact with the AI-based user guidance large language model as shown at 228, which in this example may not be shared with the user's community by default. The AI-based user guidance is in a further example presented as a chat or conversation, such as using Grok, OpenAI's ChatGPT, or another generative pre-trained transformer to conversationally provide guidance to the user based on their condition or concerns, known history, and learned or trained knowledge of the user's expressed concerns or questions.

The condition or concern in some examples may be a psychological condition, such as stress management, anxiety, depression, panic disorder, phobias, or other such conditions. In other examples, the known conditions may include weight management, exercise goals, or other physical conditions. In some such examples, counseling or guidance may be provided to assist with physical conditions such as a physical injury or abnormality. In some examples, the counseling or guidance may include performing CPR in the case of a heart attack, how to perform defibrillation using an Automated External Defibrillator (AED) in the event of a cardiac fibrillation, or how to provide first aid for another such event. In a more detailed example, guidance provided at 228 may include how to perform CPR properly and the proper pace to apply breaths and/or chest compressions. In another example, guidance provided at 228 may include guiding a person with low blood oxygen to breathe deeply, move to an environment with fresh air, and seek medical attention. Physical conditions such as these in some examples may be indicated or monitored via an external device such as a smart watch or other device operable to interact with the personal guidance application.

The guidance provided at 228 may in various examples be made available or presented to the user, and/or in various embodiments to the user's parent, guardian, caregiver, or other such user. If the user is determined to be at high risk, such as being unable to cope with their problems with the aid of the AI-based user guidance provided at 228, live person counseling may be provided at 230. In further examples, the user may perform other activities once logged in, such as journaling or recording their problems or concerns, progress toward one or more goals, and the like.

The example of FIG. 2 illustrates how a personal guidance system such as that shown in FIG. 1 can provide a user with an environment in which they can receive support from other users as well as from AI-based tools such as a large language model or generative pretrained transformer that has knowledge of their concerns and goals. The large language model or generative pretrained transformer tools may engage with the user in a conversational manner, providing guidance or counseling that is known to be relevant to their concerns and goals based on pretraining.

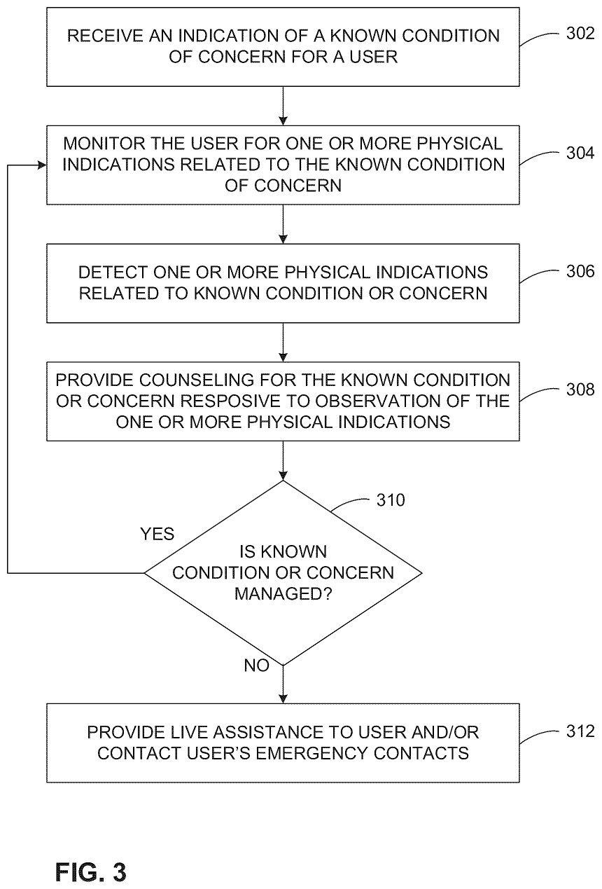

FIG. 3 is a flow diagram of a method of providing guidance to a user, consistent with an example embodiment. At 302, a personal guidance system such as that of the examples in FIGS. 1 and 2 receives an indication of a known condition of concern for a user. The known concern in some examples may be stress management, anxiety, depression, panic disorder, phobias, or other such conditions. In further examples, the known conditions may include weight management, exercise goals, or other physical conditions. The personal guidance system monitors the user at 304, such as via a smart watch having one or more biologic sensors, for physical indications related to the known condition or concern. In further examples, the biologic sensors comprise one or more of a heart rate monitor, a motion sensor, a temperature monitor, a blood oxygen monitor, a respiratory rate monitor, a speech monitor, or similar biologic or physiologic monitoring means. In some embodiments, other capture devices such as smart watches, Google Glasses, or other wearable tech may be employed as biologic or other input devices. The personal guidance system in some embodiments may similarly monitor for unknown conditions that may be indicated by the physical indications.

When one or more physical indications related to the known condition or concern are detected at 306, the personal guidance system automatically provides an indication to the user that the indications have been detected, and provides counseling for the known condition or concern at 308. If the counseling is effective in managing the known condition or concern as determined at 310, the personal guidance system may resume monitoring the user for physical indications. If the counseling provided at 308 is not effective, the personal guidance system may alert a person to provide live assistance to the user at 312, such as by connecting the user to a support person associated with the personal guidance system or by connecting the user with an emergency contact.

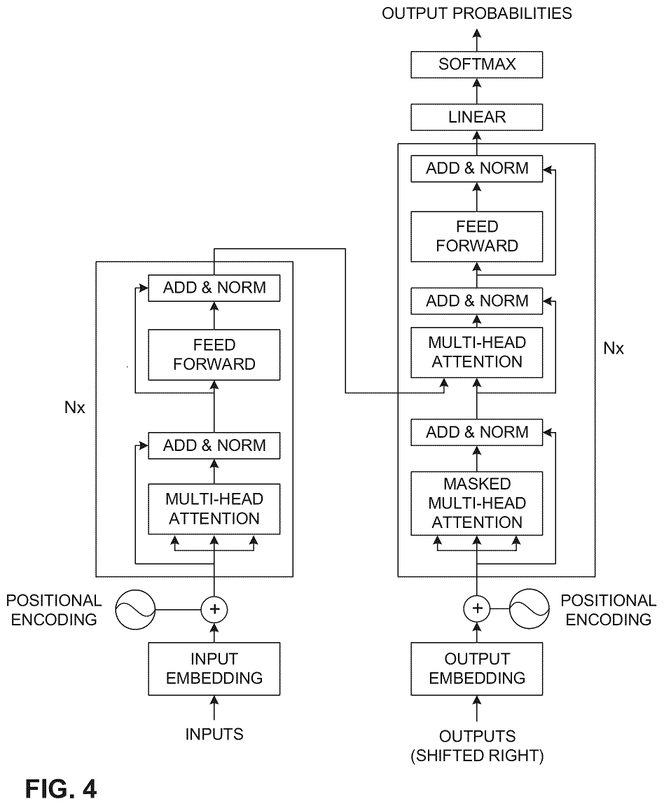

Some elements of the personal guidance system, such as the large language model-based user guidance provided in examples presented herein, may employ a generative pretrained transformer or similar technology to facilitate automated conversational interaction and guidance for a user. One example generative pretrained transformer is ChatGPT, which is shown in the block diagram of FIG. 4, but in other examples other generative pretrained transformers, recurrent neural networks, or other such technologies such as Microsoft Copilot, Google Bard, or Amazon Q may be employed. The inputs here comprise a series of words that are preprocessed (e.g. converted to numbers or other input vectors) and provided in sequence to generate output probabilities of the next word. Once the next word is obtained, it may be added to the input so that the subsequent word may be obtained, causing the ChatGPT system to repeatedly predict the next word in a response to a prompt. In a more sophisticated example, the input sequence is fixed at some value, such as 2048 words, and the extra positions at the beginning are padded with zeros. The output is similarly an array of possible outcomes with associated probabilities, such that the most probable next word may be selected as the next word in the response or output.

Because input vectors in this example indicate only a single word and comprise many more zeros than ones (e.g., ChatGPT has a vocabulary of over 50,000 input words and associated vectors), the input is embedded or encoded into a smaller multidimensional space at the input embedding element. The position of each resulting token in a sequence of inputs is encoded and provided to multi-head attention element operable to predict the degree to which each input token is likely to impact the output. The feed-forward block is a multi-layer neural network, operable to learn over time to predict the next word in a sequence. Add & norm blocks take the output of a feed-forward network block and add it to its output, which is normalized before being output. Implementation of large language models, generative pretrained transformers, and similar artificial intelligence is significantly more complex that the basic blocks described here, and any such iteration, variation, or improvement on such technology may be used in various embodiments.

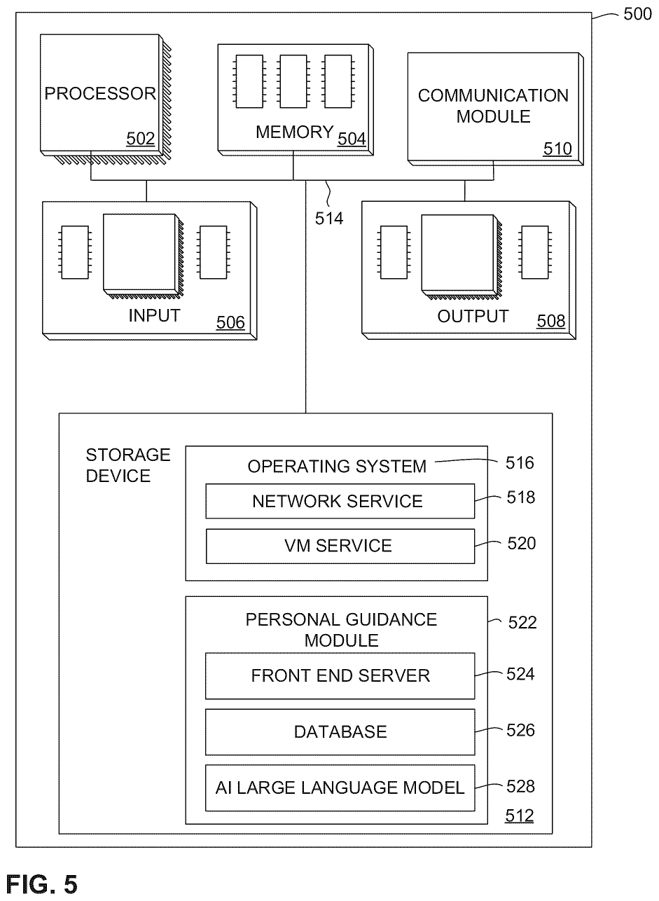

FIG. 5 shows a block diagram of a general-purpose computerized system, consistent with an example embodiment. FIG. 5 illustrates only one particular example of computing device 500, and other computing devices 500 may be used in other embodiments. Although computing device 500 is shown as a standalone computing device, computing device 500 may be any component or system that includes one or more processors or another suitable computing environment for executing software instructions in other examples, and need not include all of the elements shown here.

As shown in the specific example of FIG. 5, computing device 500 includes one or more processors 502, memory 504, one or more input devices 506, one or more output devices 508, one or more communication modules 510, and one or more storage devices 512. Computing device 500, in one example, further includes an operating system 516 executable by computing device 500. The operating system includes in various examples services such as a network service 518 and a virtual machine service 520 such as a virtual server. One or more applications, such as personal guidance module 522 are also stored on storage device 512, and are executable by computing device 500.

Each of components 502, 504, 506, 508, 510, and 512 may be interconnected (physically, communicatively, and/or operatively) for inter-component communications, such as via one or more communications channels 514. In some examples, communication channels 514 include a system bus, network connection, inter-processor communication network, or any other channel for communicating data. Applications such as personal guidance module 522 and operating system 516 may also communicate information with one another as well as with other components in computing device 500.

Processors 502, in one example, are configured to implement functionality and/or process instructions for execution within computing device 500. For example, processors 502 may be capable of processing instructions stored in storage device 512 or memory 504. Examples of processors 502 include any one or more of a microprocessor, a controller, a central processing unit (CPU), a graphics processing unit (GPU), a neural processing unit (NPU), an image signal processor (ISP), a digital signal processor (DSP), an application specific integrated circuit (ASIC), a field-programmable gate array (FPGA), or similar discrete or integrated logic circuitry.

One or more storage devices 512 may be configured to store information within computing device 500 during operation. Storage device 512, in some examples, is known as a computer-readable storage medium. In some examples, storage device 512 comprises temporary memory, meaning that a primary purpose of storage device 512 is not long-term storage. Storage device 512 in some examples is a volatile memory, meaning that storage device 512 does not maintain stored contents when computing device 500 is turned off. In other examples, data is loaded from storage device 512 into memory 504 during operation. Examples of volatile memories include random access memories (RAM), dynamic random access memories (DRAM), static random access memories (SRAM), and other forms of volatile memories known in the art. In some examples, storage device 512 is used to store program instructions for execution by processors 502. Storage device 512 and memory 504, in various examples, are used by software or applications running on computing device 500 such as personal guidance module 522 to temporarily store information during program execution.

Storage device 512, in some examples, includes one or more computer-readable storage media that may be configured to store larger amounts of information than volatile memory. Storage device 512 may further be configured for long-term storage of information. In some examples, storage devices 512 include non-volatile storage elements. Examples of such non-volatile storage elements include magnetic hard discs, optical discs, floppy discs, flash memories, or forms of electrically programmable memories (EPROM) or electrically erasable and programmable (EEPROM) memories.

Computing device 500, in some examples, also includes one or more communication modules 510. Computing device 500 in one example uses communication module 610 to communicate with external devices via one or more networks, such as one or more wireless networks. Communication module 510 may be a network interface card, such as an Ethernet card, an optical transceiver, a radio frequency transceiver, or any other type of device that can send and/or receive information. Other examples of such network interfaces include Bluetooth, 4G, LTE, or 5G, WiFi radios, and Near-Field Communications (NFC), and Universal Serial Bus (USB). In some examples, computing device 500 uses communication module 510 to wirelessly communicate with an external device such as via a public network.

Computing device 500 also includes in one example one or more input devices 506. Input device 506, in some examples, is configured to receive input from a user through tactile, audio, or video input. Examples of input device 506 include a touchscreen display, a mouse, a keyboard, a voice responsive system, video camera, microphone or any other type of device for detecting input from a user.

One or more output devices 508 may also be included in computing device 500. Output device 508, in some examples, is configured to provide output to a user using tactile, audio, or video stimuli. Output device 508, in one example, includes a display, a sound card, a video graphics adapter card, or any other type of device for converting a signal into an appropriate form understandable to humans or machines. Additional examples of output device 1008 include a speaker, a light-emitting diode (LED) display, a liquid crystal display (LCD or OLED), or any other type of device that can generate output to a user.

Computing device 500 may include operating system 516. Operating system 516, in some examples, controls the operation of components of computing device 500, and provides an interface from various applications such as personal guidance module 522 to components of computing device 500. For example, operating system 516, in one example, facilitates the communication of various applications such as personal guidance module 522 with processors 502, communication unit 510, storage device 512, input device 506, and output device 508. Applications such as personal guidance module 522 may include program instructions and/or data that are executable by computing device 500, such as front end server 524, database 526, and AI large language module 528. These and other program instructions or modules may include instructions that cause computing device 500 to perform one or more of the other operations and actions described in the examples presented herein.

Features of example computing devices employed in example embodiments may comprise features, for example, of a client computing device and/or a server computing device. The term computing device, in general, whether employed as a client and/or as a server, or otherwise, refers at least to a processor and a memory connected by a communication bus. A “processor” and/or “processing circuit” for example, is understood to connote a specific structure such as a central processing unit (CPU), digital signal processor (DSP), graphics processing unit (GPU), image signal processor (ISP) and/or neural processing unit (NPU), or a combination thereof, of a computing device which may include a control unit and an execution unit. In an aspect, a processor and/or processing circuit may comprise a device that fetches, interprets and executes instructions to process input signals to provide output signals. As such, in the context of the present patent application at least, this is understood to refer to sufficient structure within the meaning of 35 USC § 112 (f) so that it is specifically intended that 35 USC § 112 (f) not be implicated by use of the term “computing device,” “processor,” “processing unit,” “processing circuit” and/or similar terms; however, if it is determined, for some reason not immediately apparent, that the foregoing understanding cannot stand and that 35 USC § 112 (f), therefore, necessarily is implicated by the use of the term “computing device” and/or similar terms, then, it is intended, pursuant to that statutory section, that corresponding structure, material and/or acts for performing one or more functions be understood and be interpreted to be described at least in FIG. 1 and in the text associated with the foregoing figure(s) of the present patent application.

The term electronic file and/or the term electronic document, as applied herein, refer to a set of stored memory states and/or a set of physical signals associated in a manner so as to thereby at least logically form a file (e.g., electronic) and/or an electronic document. That is, it is not meant to implicitly reference a particular syntax, format and/or approach used, for example, with respect to a set of associated memory states and/or a set of associated physical signals. If a particular type of file storage format and/or syntax, for example, is intended, it is referenced expressly. It is further noted an association of memory states, for example, may be in a logical sense and not necessarily in a tangible, physical sense. Thus, although signal and/or state components of a file and/or an electronic document, for example, are to be associated logically, storage thereof, for example, may reside in one or more different places in a tangible, physical memory, in an embodiment.

In the context of the present patent application, the terms “entry,” “electronic entry,” “document,” “electronic document,” “content,”, “digital content,” “item,” and/or similar terms are meant to refer to signals and/or states in a physical format, such as a digital signal and/or digital state format, e.g., that may be perceived by a user if displayed, played, tactilely generated, etc. and/or otherwise executed by a device, such as a digital device, including, for example, a computing device, but otherwise might not necessarily be readily perceivable by humans (e.g., if in a digital format).

Also, for one or more embodiments, an electronic document and/or electronic file may comprise a number of components. As previously indicated, in the context of the present patent application, a component is physical, but is not necessarily tangible. As an example, components with reference to an electronic document and/or electronic file, in one or more embodiments, may comprise text, for example, in the form of physical signals and/or physical states (e.g., capable of being physically displayed). Typically, memory states, for example, comprise tangible components, whereas physical signals are not necessarily tangible, although signals may become (e.g., be made) tangible, such as if appearing on a tangible display, for example, as is not uncommon. Also, for one or more embodiments, components with reference to an electronic document and/or electronic file may comprise a graphical object, such as, for example, an image, such as a digital image, and/or sub-objects, including attributes thereof, which, again, comprise physical signals and/or physical states (e.g., capable of being tangibly displayed). In an embodiment, digital content may comprise, for example, text, images, audio, video, and/or other types of electronic documents and/or electronic files, including portions thereof, for example.

Also, in the context of the present patent application, the term “parameters” (e.g., one or more parameters), “values” (e.g., one or more values), “symbols” (e.g., one or more symbols) “bits” (e.g., one or more bits), “elements” (e.g., one or more elements), “characters” (e.g., one or more characters), “numbers” (e.g., one or more numbers), “numerals” (e.g., one or more numerals) or “measurements” (e.g., one or more measurements) refer to material descriptive of a collection of signals, such as in one or more electronic documents and/or electronic files, and exist in the form of physical signals and/or physical states, such as memory states. For example, one or more parameters, values, symbols, bits, elements, characters, numbers, numerals or measurements, such as referring to one or more aspects of an electronic document and/or an electronic file comprising an image, may include, as examples, time of day at which an image was captured, latitude and longitude of an image capture device, such as a camera, for example, etc. In another example, one or more parameters, values, symbols, bits, elements, characters, numbers, numerals or measurements, relevant to digital content, such as digital content comprising a technical article, as an example, may include one or more authors, for example.

Claimed subject matter is intended to embrace meaningful, descriptive parameters, values, symbols, bits, elements, characters, numbers, numerals or measurements in any format, so long as the one or more parameters, values, symbols, bits, elements, characters, numbers, numerals or measurements comprise physical signals and/or states, which may include, as parameter, value, symbol bits, elements, characters, numbers, numerals or measurements examples, collection name (e.g., electronic file and/or electronic document identifier name), technique of creation, purpose of creation, time and date of creation, logical path if stored, coding formats (e.g., type of computer instructions, such as a markup language) and/or standards and/or specifications used so as to be protocol compliant (e.g., meaning substantially compliant and/or substantially compatible) for one or more uses, and so forth.

Various example methods and embodiments are further reflected in the numbered clauses below:

Clause 1: A method, comprising: receiving an indication of a known condition for a user; monitoring the user for one or more physical indications; and providing counseling via a large language model, including autonomous systems capable of monitoring, analyzing, and providing adaptive guidance for personal, professional, and operational contexts. Monitoring may extend to physical systems, including wearable devices, stationary computing platforms, or networked autonomous systems.

Clause 2: The method of clause 1, wherein the one or more physical indications suggest one or more physical injuries or abnormalities. Physical indications include ergonomic risk factors, environmental hazards, and physiological stress levels monitored by input or output devices. The system may incorporate predictive algorithms to identify potential abnormalities before they manifest.

Clause 3: The method of clause 2, wherein the counseling comprises first aid for the one or more physical injuries or abnormalities. The networked personal guidance system may autonomously coordinate responses, such as dispatching alerts, providing real-time guidance, or interacting with connected systems to deliver first aid and recommendations.

Clause 4: The method of claim 1, wherein the known condition comprises a psychological condition. Psychological conditions include mental health challenges linked to professional roles, social environments, and personal well-being. AI systems may analyze behavioral patterns to deliver interactive emotional support.

Clause 5: The method of clause 4, wherein the psychological condition comprises one or more of stress management, anxiety, depression, panic disorder, and phobias. Psychological conditions include role-specific stress, social isolation, performance anxiety, and adaptive coping strategies.

Clause 6: The method of clause 1, wherein monitoring comprises using a smart watch to monitor the one or more physical indications. Monitoring includes data collection via wearables, stationary systems, and other input/output devices, enabling comprehensive real-time analysis.

Clause 7: The method of clause 6, wherein providing counseling comprises providing counseling via the smart watch or a device linked to the smart watch. Counseling is delivered via networked devices, including wearable interfaces, computing systems, Bluetooth, and interactive autonomous systems.

Clause 8: The method of clause 1, wherein the large language model comprises a generative pretrained transformer. The generative pretrained transformer is configured for role-specific guidance, predictive analytics, and task automation across various user contexts. Capabilities include real-time learning and adaptation for new tasks in dynamic environments.

Clause 9: The method of clause 1, wherein the one or more physical indications comprise one or more of heart rate, movement, body temperature, blood oxygen, speech, and respiratory rate. Biometric indicators include neurophysiological metrics, behavioral responses, and stress patterns integrated into predictive health models. Inputs are collected via interconnected Bluetooth or wearable and stationary systems for holistic data analysis.

Clause 10: A method comprising: receiving an indication of a known condition for a user; receiving input from the user related to the known condition; and providing counseling for the known condition to the user responsive to the received input via a large language model trained on the condition, the large language model adapted based on the received input. Adaptations include dynamically reconfiguring algorithms to address personal, professional, or environmental contexts. Input adaptation extends to role-based modifications for specific industries, such as healthcare or logistics.

Clause 11: The method of clause 10, wherein adapting the large language model based on the received input comprises training a user-specific version of the large language model based on the received input. The system may generate user-specific models that adapt to professional roles, workflows, or collaborative environments. Training may employ secure, decentralized methods to ensure data integrity across distributed networks.

Clause 12: The method of clause 10, wherein adapting the large language model based on the received input comprises adapting a personality of the large language model based on the received input, and wherein the received input comprises a user prompt provided to the large language model. Personality adaptation may include adjustments for contextual roles, such as caregiving, customer service, or creative collaboration. The system may autonomously refine its tone and or voice interaction style to align with user preferences.

Clause 13: The method of clause 10, wherein the large language model is further operable to remember a state of prior interactions with the user. Memory retention may support task histories, project logs, and longitudinal analysis for multi-session workflows. Interaction data may be stored securely and retrieved dynamically for context-aware continuity.

Clause 14: The method of clause 13, wherein the remembered state of prior interactions with a user enable the large language model to provide consistent conversational guidance across multiple login sessions for the user. Consistent guidance may extend across platforms and devices, enabling seamless transitions between wearables, stationary systems, Bluetooth, and autonomous interfaces.

Clause 15: The method of clause 10, further comprising providing a reference or a factual basis for the provided counseling. References may include real-time contextual data sourced from environmental, occupational, or health-related inputs. The system may verify sources dynamically to ensure accuracy and relevance.

Clause 16: A computing apparatus, comprising: a processor and a memory; a large language model stored in the memory and operable to execute on the processor, the large language model operable to receive an indication of a known condition for a user, to receive input from the user related to the known condition, and to provide counseling for the known condition to the user responsive to the received input via a large language model trained on the condition. The apparatus may support physical embodiments, including stationary systems, Bluetooth and networked autonomous devices equipped with large language models.

Clause 17: The apparatus of clause 16, the computing apparatus further comprising an input operable to monitor the user for one or more physical indications related to the known condition. The input may extend to multimodal systems, including gesture-based interfaces, neural sensors, Bluetooth, and interactive robotics.

Clause 18: The apparatus of clause 17, wherein the input comprises a smart watch. Adaptations may include real-time operational reconfigurations for task-specific execution, collaboration, and environmental changes.

Clause 19: The apparatus of clause 16, wherein the large language model is further adapted based on the received input from the user. Interaction memory may be used to maintain context continuity across diverse workflows and platforms.

Clause 20: The apparatus of clause 19, wherein the large language model is further operable to remember a state of prior interactions with the user to provide consistent conversational guidance across multiple login sessions for the user. Guidance may incorporate cross-device synchronization and adaptive workflows to optimize user experience across sessions.

Although specific embodiments have been illustrated and described herein, any arrangement that achieve the same purpose, structure, or function may be substituted for the specific embodiments shown. This application is intended to cover any adaptations or variations of the example embodiments of the invention described herein. These and other embodiments are within the scope of the following claims and their equivalents.

What is claimed is:

1. A method comprising:
receiving an indication of a known condition for a user;
monitoring the user for one or more physical indications related to the known condition; and
providing counseling for the known condition to the user responsive to observation of the one or more monitored physical indications via a large language model trained on the condition.

2. The method of claim 1, wherein the one or more physical indications suggest one or more physical injuries or abnormalities.

3. The method of claim 2, wherein the counseling comprises first aid for the one or more physical injuries or abnormalities.

4. The method of claim 1, wherein the known condition comprises a psychological condition.

5. The method of claim 4, wherein the psychological condition comprises one or more of stress management, anxiety, depression, panic disorder, or phobias.

6. The method of claim 1, wherein monitoring comprises using a smart watch to monitor the one or more physical indications.

7. The method of claim 6, wherein providing counseling comprises providing counseling via the smart watch or a device linked to the smart watch.

8. The method of claim 1, wherein the large language model comprises a generative pretrained transformer operable to provide the large language model with contextual understanding such that the large language model may provide a relevant response to a variety of inputs.

9. The method of claim 1, wherein the one or more physical indications comprise one or more of heart rate, movement, body temperature, blood oxygen, speech, or respiratory rate.

10. A method comprising:
receiving an indication of a known condition for a user;
receiving input from the user related to the known condition; and
providing counseling for the known condition to the user responsive to the received input via a large language model trained on the condition, the large language model adapted based on the received input.

11. The method of claim 10, wherein adapting the large language model based on the received input comprises training a user-specific version of the large language model based on the received input.

12. The method of claim 10, wherein adapting the large language model based on the received input comprises adapting a personality of the large language model based on the received input, and wherein the received input comprises a user prompt provided to the large language model.

13. The method of claim 10, wherein the large language model is further operable to remember a state of prior interactions with the user.

14. The method of claim 13, wherein the remembered state of prior interactions with a user enable the large language model to provide consistent conversational guidance across multiple login sessions for the user.

15. The method of claim 10, further comprising providing a reference or a factual basis for the provided counseling.

16. A computing apparatus, comprising:
a processor and a memory;
a large language model stored in the memory and operable to execute on the processor, the large language model operable to receive an indication of a known condition for a user, to receive input from the user related to the known condition, and to provide counseling for the known condition to the user responsive to the received input via a large language model trained on the condition.

17. The apparatus of claim 16, the computing apparatus further comprising an input operable to monitor the user for one or more physical indications related to the known condition.

18. The apparatus of claim 17, wherein the input comprises a smart watch.

19. The apparatus of claim 16, wherein the large language model is further adapted based on the received input from the user.

20. The apparatus of claim 19, wherein the large language model is further operable to remember a state of prior interactions with the user to provide consistent conversational guidance across multiple login sessions for the user.
